(19) United States
(12) Patent Application Publication
(10) Pub. No.: US 2026/0042720 A1
(43) Pub. Date: Feb. 12, 2026

(54) AZEOTROPE OR AZEOTROPE-LIKE COMPOSITIONS OF 1,1,2,2,3-PENTACHLOROPROPANE AND HYDROGEN FLUORIDE (HF) AND METHODS FOR PRODUCING TRIFLUOROCHLOROPROPENES

(71) Applicant: Honeywell International Inc., Charlotte, NC (US)

(72) Inventors: Haiyou Wang, Amherst, NY (US); Haluk Kopkalli, Staten Island, NY (US); Hang T. Pham, Amherst, NY (US); Terris Yang, East Amherst, NY (US); Joshua Close, Cheektowaga, NY (US); Daniel C. Merkel, Orchard Park, NY (US); Chenguang Liang, Niagara Falls (CA); Yang Luo, East Amherst, NY (US)

(21) Appl. No.: 19/280,751

(22) Filed: Jul. 25, 2025

Related U.S. Application Data
(60) Provisional application No. 63/681,403, filed on Aug. 9, 2024.

(51) Int. Cl. C07C 17/20; B01J 21/04; B01J 27/08; B01J 27/132; C07B 39/00

(52) U.S. Cl. CPC C07C 17/208; B01J 21/04; B01J 27/08; B01J 27/132; C07B 39/00

(57) ABSTRACT

Minimum-boiling, heterogeneous azeotropic and azeotrope-like compositions including 1,1,2,2,3-pentachloropropane (HCC-240aa) and hydrogen fluoride (HF), as well as methods for using these azeotropic or azeotrope-like compositions in the production of trifluorochloropropenes.

### CROSS-REFERENCE TO RELATED APPLICATION

This application claims the benefit under 35 U.S.C. § 119 (e) of U.S. U.S. Provisional Patent Application Ser. No. 63/681,403, filed on Aug. 9, 2024, the entire disclosure of which is incorporated by reference herein.

### FIELD

The present disclosure is related to azeotrope or azeotrope-like compositions comprising 1,1,2,2,3-pentachloropropane (HCC-240aa) and hydrogen fluoride (HF), as well as methods for exploiting or resolving the azeotropic and azeotrope-like mixtures when encountered in a process for producing trifluorochloropropenes.

### BACKGROUND

Fluorocarbon fluids have properties that are desirable for use as heat transfer media, immersion coolants, liquid or gaseous dielectrics, industrial refrigerants, and other applications. For these applications, the use of single component fluids or azeotrope-like mixtures, i.e., those which do not substantially fractionate on boiling and evaporation, are particularly desirable.

However, the use of certain hydrofluorocarbons “HFCs” in industrial applications is now believed to contribute to global warming, and accordingly, their contemporary use has been curtailed.

The identification of new, environmentally safe, non-fractionating mixtures comprising HFCs is complicated, because azeotrope formation is not readily predictable. Therefore, the industry is continually seeking new HFC-based mixtures that are acceptable and environmentally safer substitutes.

### SUMMARY

It has been found that certain azeotropic and azeotrope-like compositions may arise from mixtures of 1,1,2,2,3-pentachloropropane (HCC-240aa) and hydrogen fluoride (HF). In particular, the present disclosure provides minimum-boiling, heterogeneous azeotropic or azeotrope-like compositions consisting essentially of 1,1,2,2,3-pentachloropropane (HCC-240aa) and hydrogen fluoride (HF).

In addition, the present disclosure provides methods for exploiting or resolving the azeotropic and azeotrope-like mixtures when encountered in a process for producing trifluorochloropropenes.

In one form thereof, a composition is provided which may comprise an azeotrope or azeotrope-like composition consisting essentially of effective amounts of 1,1,2,2,3-pentachloropropane (HCC-240aa) and hydrogen fluoride (HF), wherein the azeotrope or azeotrope-like composition has a boiling point of about 19° C. at a pressure of about 14.7 psia±0.2 psia.

In another form thereof, a process to produce trifluorochloropropenes is provided where such process comprising the steps of: providing a reactant composition comprising 1,1,2,2,3-pentachloropropane (HCC-240aa); and fluorinating the 1,1,2,2,3-pentachloropropane (HCC-240aa) with hydrogen fluoride (HF) and a catalyst to produce a product mixture.

In yet another form thereof, a process for the production of trifluorochloropropenes is provided, the process comprising the steps of: providing a reactant composition comprising 1,1,2,2,3-pentachloropropane (HCC-240aa); fluorinating the 1,1,2,2,3-pentachloropropane (HCC-240aa) with hydrogen fluoride (HF) and a catalyst to produce a product mixture comprising an azeotrope or azeotrope-like composition of 1,1,2,2,3-pentachloropropane (HCC-240aa) and hydrogen fluoride (HF); separating the azeotrope or azeotrope-like composition of 1,1,2,2,3-pentachloropropane (HCC-240aa) and hydrogen fluoride (HF) from the product mixture; and recycling the azeotrope or azeotrope-like composition back into the reactant composition.

### DESCRIPTION OF THE DRAWINGS

FIG. 1 illustrates P-T-X data of a mixture of 1,1,2,2,3-pentachloropropane (HCC-240aa) and hydrogen fluoride (HF) at 19.8° C.

FIG. 2 illustrates P-T-X data of a mixture of 1,1,2,2,3-pentachloropropane (HCC-240aa) and hydrogen fluoride (HF) at 39.8° C.

FIG. 3 illustrates P-T-X data of a mixture of 1,1,2,2,3-pentachloropropane (HCC-240aa) and hydrogen fluoride (HF) at 59.8° C.

FIG. 4 is a schematic illustrating a process for producing trifluorochloropropenes from azeotropic or azeotrope-like compositions consisting essentially of 1,1,2,2,3-pentachloropropane (HCC-240aa) and hydrogen fluoride (HF).

### DETAILED DESCRIPTION

### I. Definitions and Description of Azeotrope or Azeotrope-Like Compositions

An “azeotrope” (or “azeotropic”) composition is a unique combination of two or more components. An azeotrope can be either homogenous (which has one liquid phase) or heterogeneous (which has two liquid phases). An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). However, in the case of a heterogeneous azeotrope the boiling point of the azeotrope will always be below the boiling point of the lower boiling point component. In the case of a heterogeneous azeotrope then at this characteristic temperature the composition of each of the two liquid phases and the vapor phase will remain constant upon boiling. The azeotrope composition does not fractionate upon boiling or evaporation. Therefore, the components of the azeotrope composition cannot be separated during a phase change.

A heterogeneous azeotrope consists of two liquid phases and one vapor phase, or one solid, one liquid, and one vapor phase, all in equilibrium. For a heterogeneous azeotrope at a given temperature and pressure, the composition of each of the two liquid phases and the composition of the vapor phase remain constant. If a heterogeneous azeotrope is formed, at a constant pressure the boiling point of the heterogeneous azeotrope will be less than the lower boiling point component (a “minimum boiling azeotrope”).

Thus, stated further, an “azeotrope” composition is a unique combination of two or more components for which compositions of vapor and liquids are the same which yield and minimum or maximum in saturation temperature or pressure. As an extension, azeotrope can be classified as being homogeneous or heterogeneous. According to Seader and Henley (Separation Process Principles, Wiley, Second Edition, 2006, pp. 123-126), if only one liquid phase exists, the mixture forms a homogeneous azeotrope; if more than one liquid is present, the azeotrope is heterogeneous. For a fixed temperature, heterogeneous azeotropes, according to Seader and Henley, have total pressures and phase compositions that remain constant across the multiphase region (a region that is sometimes referred to as the “miscibility gap”). In contrast, for a fixed temperature, homogeneous azeotropes yield only one unique total pressure where phase compositions are constant given that a multiphase region is, by definition, absent. While both heterogeneous and homogeneous azeotropes share features of constant composition, the presence or absence of a liquid multiphase region yields a difference that permits or prevents their application, use, and/or otherwise treatment of the resulting azeotrope; therefore, distinguishing an azeotrope as homogeneous or heterogeneous becomes critically important and necessary for their application, use, and/or otherwise treatment.

An azeotrope composition is also characterized in that at the characteristic azeotrope temperature, the bubble point pressure of the liquid phase is identical to the dew point pressure of the vapor phase.

The behavior of an azeotrope composition is in contrast with that of a non-azeotrope composition in which during boiling or evaporation, the liquid composition changes to a substantial degree.

For the purposes of the present disclosure, an azeotrope composition is characterized as that composition which boils at a constant characteristic temperature, the temperature being lower (a minimum boiling azeotrope) than the boiling points of the two or more components, and thereby having the same composition in both the vapor and liquid phases.

One of ordinary skill in the art would understand that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

An “azeotrope-like” composition is a composition of two, three, or more components which behaves substantially as an azeotrope composition. Thus, for the purposes of this disclosure, an azeotrope-like composition is a combination of two, three or more different components which, when in liquid form under given pressure, will boil at a substantially constant temperature, and which will provide a vapor composition substantially identical to the liquid composition undergoing boiling.

Azeotrope or azeotrope-like compositions can be identified by several different methods.

Static Vapor-Liquid Equilibrium Methods are a class of experimental techniques that can also be used to identify the presence of azeotrope and azeotrope-like compositions. One such technique, known as the PTx method, collects measurements of the total saturation pressure (“P”) exerted by mixtures of known compositions (“x”) at fixed temperatures (“T”) and cell volumes. (Walas, Phase Equilibria in Chemical Engineering, Butterworth-Heinemann, 1985, pp. 537). Using data collected from the PTx experiment, as well as pure component properties of constituents of the mixtures, the thermodynamic properties of the mixture can be accurately characterized by fitting the component's interaction parameters in a well-defined thermodynamic equation; one such equation is the Non-random, Two-Liquid (NRTL) activity coefficient model described by Renon and Prausnitz (Local Compositions in Thermodynamic Excess Functions for Liquid Mixtures, AlChE Journal, Vol. 14, January 1968, pp. 135-144).

The presence of an azeotrope and its corresponding composition can be observed by plotting saturation pressure measurements from PTx data and saturation pressures described by NRTL as a function of composition. For a given temperature (isotherm), the presence of an azeotrope composition is identified by the observation of a maximum or minimum in total pressure that is greater or less than the pure saturation pressures of any of the components alone.

The boiling points of each of the components alone are measured at a constant pressure. As the skilled person will appreciate, for a binary azeotrope or azeotrope-like composition, the boiling point of one of the components of the composition is initially measured. The second component of the composition is then added in varying amounts and the boiling point of each of the obtained compositions is measured using the ebulliometer at said constant pressure.

The measured boiling points are plotted against the composition of the tested composition, for example, for a binary azeotrope, the amount of the second component added to the composition, (expressed as either mass or weight %, wt. %, or mole %). The presence of an azeotrope composition can be identified by the observation of a maximum or minimum boiling temperature which is greater or less than the boiling points of any of the components alone.

As the skilled person will appreciate, the identification of the azeotrope or azeotrope-like composition is made by the comparison of the change in the boiling point of the composition on addition of the second component to the first component, relative to the boiling point of the first component. Thus, it is not necessary that the system be calibrated to the reported boiling point of the particular components in order to measure the change in boiling point.

As used herein, the term “1,1,2,2,3-pentachloropropane” refers to HCC-240aa which may be abbreviated as R-240aa.

As used herein, the term “consisting essentially of”, with respect to the components of an azeotrope or azeotrope-like composition or mixture, means the composition contains the indicated components in an azeotrope or azeotrope-like ratio, and may contain additional components provided that the additional components do not form new azeotrope or azeotrope-like systems. For example, azeotrope mixtures consisting essentially of two compounds are those that form binary azeotropes, which optionally may include one or more additional components, provided that the additional components do not render the mixture non-azeotropic and do not form an azeotrope with either or both of the compounds (e.g., do not form a ternary or higher azeotrope).

As used herein, the term “about”, when used in connection with recited weight percentages of the components of the present compositions, includes a deviation of ±0.3% from the recited weight percentage.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

As used herein, the term “effective amount” is an amount of each component which, when combined with the other component, results in the formation of an azeotrope or azeotrope-like mixture.

As previously discussed, at the maximum or minimum boiling point, the composition of the vapor phase will be identical to the composition of the liquid phase. The azeotrope-like composition is therefore that composition of components which provides a substantially constant minimum or maximum boiling point at which substantially constant boiling point the composition of the vapor phase will be substantially identical to the composition of the liquid phase.

II. Azeotrope or Azeotrope-Like Compositions of 1,1,2,2,3-pentachloropropane (HCC-240aa) and Hydrogen Fluoride (HF)

It has been found that 1,1,2,2,3-pentachloropropane (HCC-240aa) and hydrogen fluoride (HF) form a heterogeneous, minimum boiling azeotrope or azeotrope-like composition, and the present disclosure provides heterogeneous azeotrope or azeotrope-like compositions comprising 1,1,2,2,3-pentachloropropane (HCC-240aa) and hydrogen fluoride (HF). The azeotrope or azeotrope-like compositions may comprise 1,1,2,2,3-pentachloropropane (HCC-240aa) and hydrogen fluoride (HF). The azeotrope or azeotrope-like compositions may consist essentially of 1,1,2,2,3-pentachloropropane (HCC-240aa) and hydrogen fluoride (HF). The azeotrope or azeotrope-like compositions may consist of 1,1,2,2,3-pentachloropropane (HCC-240aa) and hydrogen fluoride (HF).

The present inventors have found experimentally that 1,1,2,2,3-pentachloropropane (HCC-240aa) and hydrogen fluoride (HF) form an azeotrope or azeotrope-like composition.

The azeotrope or azeotrope-like composition of 1,1,2,2,3-pentachloropropane (HCC-240aa) and hydrogen fluoride (HF) may be a binary azeotrope, which includes only the foregoing two components and lacks other components.

The present disclosure provides an azeotrope or azeotrope-like composition which comprises effective amounts of 1,1,2,2,3-pentachloropropane (HCC-240aa) and hydrogen fluoride (HF) to form an azeotrope or azeotrope-like composition.

The present azeotrope or azeotrope-like compositions may consist essentially of combinations of 1,1,2,2,3-pentachloropropane (HCC-240aa) and hydrogen fluoride (HF).

The present azeotrope or azeotrope-like compositions may consist of combinations of 1,1,2,2,3-pentachloropropane (HCC-240aa) and hydrogen fluoride (HF).

The present disclosure also provides a method of forming an azeotrope or azeotrope-like composition by contacting, mixing, combining, or blending, effective amounts of 1,1,2,2,3-pentachloropropane (HCC-240aa) and hydrogen fluoride (HF). Any of a wide variety of methods known in the art for combining two or more components to form a composition can be used in the present methods. For example, 1,1,2,2,3-pentachloropropane (HCC-240aa) and hydrogen fluoride (HF) can be mixed, blended, or otherwise combined by hand and/or by machine, as part of a batch or continuous reaction and/or process, or via combinations of two or more such steps. Both 1,1,2,2,3-pentachloropropane (HCC-240aa) and hydrogen fluoride (HF) are commercially available and can be procured from several different vendors. The components can be provided in the required amounts, for example by weighing and then combining the amounts.

Preferably, the azeotrope or azeotrope-like composition may comprise from about 0.1 wt. % to about 78.9 wt. % 1,1,2,2,3-pentachloropropane (HCC-240aa), preferably from about 0.2 wt. % to about 72.5 wt. % 1,1,2,2,3-pentachloropropane (HCC-240aa), or preferably from about 0.6 wt. % to about 64.0 wt. % 1,1,2,2,3-pentachloropropane (HCC-240aa) and from about 21.1 wt. % to about 99.9 wt. % hydrogen fluoride (HF), preferably from about 27.5 wt. % to about 99.8 wt. % hydrogen fluoride (HF), or preferably from about 36.0 wt. % to about 99.4 wt. % hydrogen fluoride (HF).

Preferably, the azeotrope or azeotrope-like composition may consist essentially of from about 0.1 wt. % to about 78.9 wt. % 1,1,2,2,3-pentachloropropane (HCC-240aa), preferably from about 0.2 wt. % to about 72.5 wt. % 1,1,2,2,3-pentachloropropane (HCC-240aa), or preferably from about 0.6 wt. % to about 64.0 wt. % 1,1,2,2,3-pentachloropropane (HCC-240aa) and from about 21.1 wt. % to about 99.9 wt. % hydrogen fluoride (HF), preferably from about 27.5 wt. % to about 99.8 wt. % hydrogen fluoride (HF), or preferably from about 36.0 wt. % to about 99.4 wt. % hydrogen fluoride (HF).

Preferably, the azeotrope or azeotrope-like composition may consist of from about 0.1 wt. % to about 78.9 wt. % 1,1,2,2,3-pentachloropropane (HCC-240aa), preferably from about 0.2 wt. % to about 72.5 wt. % 1,1,2,2,3-pentachloropropane (HCC-240aa), or preferably from about 0.6 wt. % to about 64.0 wt. % 1,1,2,2,3-pentachloropropane (HCC-240aa) and from about 21.1 wt. % to about 99.9 wt. % hydrogen fluoride (HF), preferably from about 27.5 wt. % to about 99.8 wt. % hydrogen fluoride (HF), or preferably from about 36.0 wt. % to about 99.4 wt. % hydrogen fluoride (HF).

The azeotrope or azeotrope-like composition may also comprise about 9.5 wt. % 1,1,2,2,3-pentachloropropane (HCC-240aa) and about 90.5 wt. % hydrogen fluoride (HF). The azeotrope or azeotrope-like composition may also consist essentially of about 9.5 wt. % 1,1,2,2,3-pentachloropropane (HCC-240aa) and about 90.5 wt. % hydrogen fluoride (HF). The azeotrope or azeotrope-like composition may also consist of about 9.5 wt. % 1,1,2,2,3-pentachloropropane (HCC-240aa) and about 90.5 wt. % hydrogen fluoride (HF). Preferably, the azeotrope or azeotrope-like composition of the present disclosure has a boiling point of about 19° C. at a pressure of about 14.7 psia±0.2 psia.

In other words, the azeotrope or azeotrope-like composition comprise from about 0.1 wt. % to 78.9 wt. % of 1,1,2,2,3-pentachloropropane (HCC-240aa) and 21.1 wt. % to 99.9 wt. % hydrogen fluoride (HF), from about 0.2 wt. % to 72.5 wt. % 1,1,2,2,3-pentachloropropane (HCC-240aa) and 27.5 wt. % to 99.8 wt. % hydrogen fluoride (HF), from about 0.6 wt. % to 64.0 wt. % 1,1,2,2,3-pentachloropropane (HCC-240aa) and 36.0 wt. % to 99.4 wt. % hydrogen fluoride (HF), or about 9.5 wt. % 1,1,2,2,3-pentachloropropane (HCC-240aa) and 90.5 wt. % hydrogen fluoride (HF).

Alternatively, the azeotrope or azeotrope-like composition consist essentially of from about 0.1 wt. % to 78.9 wt. % of 1,1,2,2,3-pentachloropropane (HCC-240aa) and 21.1 wt. % to 99.9 wt. % hydrogen fluoride (HF), from about 0.2 wt. % to 72.5 wt. % 1,1,2,2,3-pentachloropropane (HCC-240aa) and 27.5 wt. % to 99.8 wt. % hydrogen fluoride (HF), from about 0.6 wt. % to 64.0 wt. % 1,1,2,2,3-pentachloropropane (HCC-240aa) and 36.0 wt. % to 99.4 wt. % hydrogen fluoride (HF), or about 9.5 wt. % 1,1,2,2,3-pentachloropropane (HCC-240aa) and 90.5 wt. % hydrogen fluoride (HF).

Alternatively, the azeotrope or azeotrope-like composition consist of from about 0.1 wt. % to 78.9 wt. % of 1,1,2,2,3-pentachloropropane (HCC-240aa) and 21.1 wt. % to 99.9 wt. % hydrogen fluoride (HF), from about 0.2 wt. % to 72.5 wt. % 1,1,2,2,3-pentachloropropane (HCC-240aa) and 27.5 wt. % to 99.8 wt. % hydrogen fluoride (HF), from about 0.6 wt. % to 64.0 wt. % 1,1,2,2,3-pentachloropropane (HCC-240aa) and 36.0 wt. % to 99.4 wt. % hydrogen fluoride (HF), or about 9.5 wt. % 1,1,2,2,3-pentachloropropane (HCC-240aa) and 90.5 wt. % hydrogen fluoride (HF).

The present disclosure also provides a composition comprising the azeotrope or azeotrope-like composition. For example, provided is a composition comprising at least about 5 wt. % of the azeotrope or azeotrope-like composition, or at least about 15 wt. % of the azeotrope or azeotrope-like composition, or at least about 50 wt. % of the azeotrope or azeotrope-like composition, or at least about 70 wt. % of the azeotrope or azeotrope-like composition, or at least about 90 wt. % of the azeotrope or azeotrope-like composition.

IV. Methods for Manufacturing Trifluorochloropropenes and/or 2,3,3,3-tetrafluoro-1-propene (HFO-1234yf) using Compositions of 1,1,2,2,3-Pentachloropropane (HCC-240aa) and Hydrogen Fluoride (HF).

The present disclosure also relates to methods for using 1,1,2,2,3-pentachloropropane (HCC-240aa) and hydrogen fluoride (HF) to produce trichlorofluoropropenes and/or difluorodichloropropenes. These methods generally include the following step of Scheme 1:

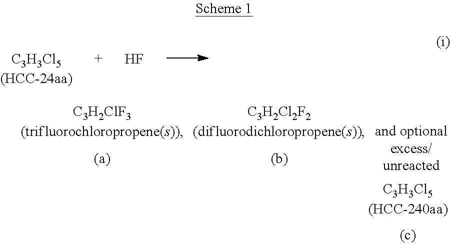

Here, in step (i), a reactant composition comprising 1,1,2,2,3-pentachloropropane (HCC-240aa) is provided and is subsequently fluorinated, in the presence of a catalyst, such as by reacting the 1,1,2,2,3-pentachloropropane (HCC-240aa) with hydrogen fluoride (HF), to produce a product mixture. Such product mixture has been found to contain any one of, or combination of (a) trifluorochloropropene(s) such as 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233yd(Z)), (b) difluorodichloropropene(s) such as 1,3-dichloro-3,3-difluoro-1-propene (HFC-1232zd) isomers, and residual/unreacted 1,1,2,2,3-pentachloropropane (HCC-240aa).

It has also been found that 2,3,3,3-tetrafluoro-1-propene (HFO-1234yf) can be produced by a further reaction process utilizing the 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), 1,3-dichloro-3,3-difluoro-1-propene (HFC-1232zd) produced in the foregoing process.

Here, the preparation of 2,3,3,3-tetrafluoro-1-propene (HFO-1234yf) generally includes the following at three reaction steps of Scheme 2:

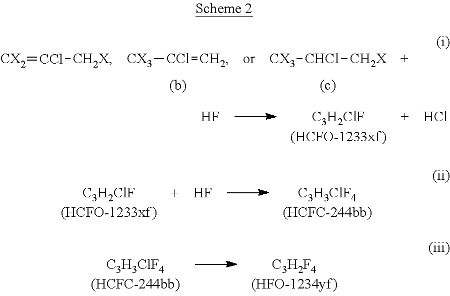

In step (i), in a vapor phase reactor charged with a catalyst, (a) CX₂═CCl—CH₂X, (b) CX₃—CCl═CH₂, or (c) CX₃—CHCl—CH₂X (where X is independently selected from F, Cl, Br, and I, provided that at least one X is not fluorine) is reacted with HF to produce 2-chloro-3,3,3-trifluoropropene (HCFO-1233xf) and HCl. In step ii) in a liquid phase reactor in the presence of a liquid hydrofluorination catalyst, the HCFO-1233xf is reacted with HF to produce 2-chloro-1,1,1,2-tetrafluoropropane (HCFC-244bb). In step (iii), in a vapor phase reactor, the HCFC-244bb is further reacted to produce HFO-1234yf.

It has been found that the 2-chloro-3,3,3-trifluoroprop-1-ene (HCFO-1233xf) and/or any one of the trans-1-chloro-3,3,3-trifluoropropene (HCFO-1233zd(E)), cis-1-chloro-3,3,3-trifluoropropene (HCFO-1233zd(Z)), and cis-1-chloro-2,3,3-trifluoroprop-1-ene (HCFO-1233 yd(Z)) can be used as an alternative and/or replacement for the 2-chloro-3,3,3-trifluoroprop-1-ene (HCFO-1233xf) used in step (i) of Scheme 2. Therefore, step (i) of Scheme 2 can be avoided/replaced with step i) of Scheme 1 when producing 2,3,3,3-tetrafluoro-I-propene (HFO-1234yf), as provided in Scheme 3:

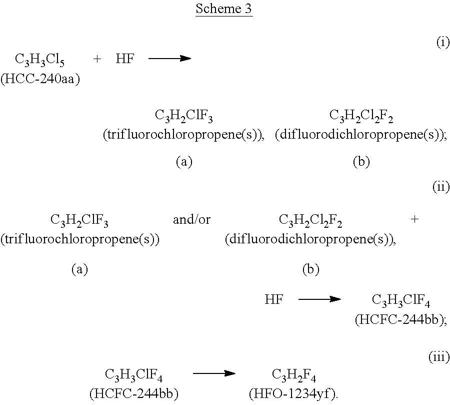

In step (i), in step (i), a reactant composition comprising 1,1,2,2,3-pentachloropropane (HCC-240aa) is provided and is fluorinated, in the presence of a catalyst, such as by reacting the 1,1,2,2,3-pentachloropropane (HCC-240aa) with hydrogen fluoride (HF), to produce a product mixture. The product mixture may comprise any one of, or combination of (a) trifluorochloropropenes including 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), and/or (b) difluorodichloropropenes including 1,3-dichloro-3,3-difluoro-1-propene (HFC-1232zd). In step ii) in a liquid phase reactor in the presence of a liquid hydrofluorination catalyst, the (a) trifluoropropenes including 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), and/or (b) difluoropropenes including 1,3-dichloro-3,3-difluoro-1-propene (HFC-1232zd) is/are reacted with HF to produce 2-chloro-1,1,1,2-tetrafluoropropane (HCFC-244bb). In step (iii), in a vapor phase reactor, the HCFC-244bb is further reacted to produce HFO-1234yf.

It has also been found that the residual azeotrope or azeotrope-like compositions of 1,1,2,2,3-pentachloropropane (HCC-240aa) and hydrogen fluoride (HF) of the product mixture may be separated from the product mixture and then recycled back into the reaction process as a reactant to be fluorinated again. The fluorination reaction may be carried out in the gas or vapor phase in a suitable reactor, for example a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers.

### Process Flow

The reactor may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow may be in the down or up direction through a bed of the catalyst. Products may be flowed through one or more scrubbers and/or distillation columns to remove by-products from the reaction, such as hydrogen fluoride (HF) and/or hydrogen chloride (HCl), and the reaction products may be collected by capture in a cooled cylinder, for example.

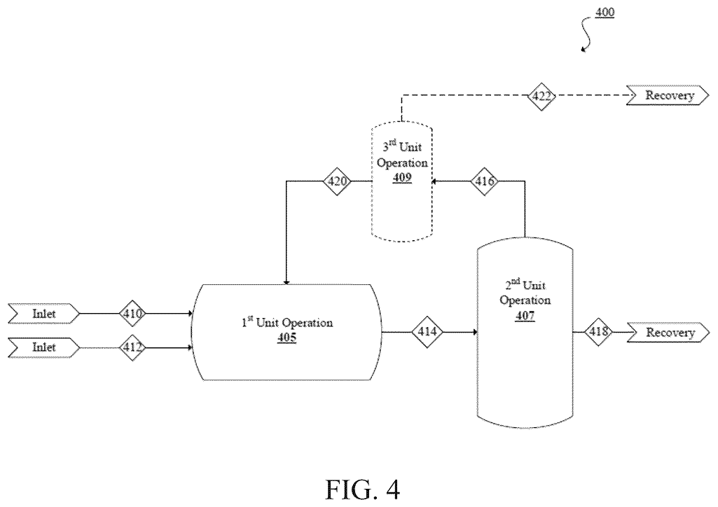

FIG. 4 is a representative process flow diagram of an exemplary reactor setup 400. Here, feed stream 410 may comprise 1,1,2,2,3-pentachloropropane (HCC-240aa) and feed stream 412 may comprise hydrogen fluoride (HF). Both feed streams 410 and 412 may comprise the same feed stream. Feed streams 410 and 412 are fed into 1st unit operation 405, which may be a reactor as described previously. Here the 1,1,2,2,3-pentachloropropane (HCC-240aa) is fluorinated with hydrogen fluoride (HF) in the presence of a catalyst, producing product stream 414. Product stream 414 comprises any one of the components of the product mixture described previously, such as 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), 1,3-dichloro-3,3-difluoro-1-propene (HFC-1232zd) isomers, and/or 1,1,2,2,3-pentachloropropane (HCC-240aa), hydrogen fluoride (HF), and the azeotrope or azeotrope like composition of 1,2,2,3-pentachloropropane (HCC-240aa) and hydrogen fluoride (HF).

Product stream 414 is then fed into 2^{nd} Unit Operation 407, which separates the 1,2,2,3-pentachloropropane (HCC-240aa), hydrogen fluoride (HF), and the azeotrope or azeotrope like composition of 1,2,2,3-pentachloropropane (HCC-240aa) and hydrogen fluoride (HF) from the other components of product stream 414. For example, 2^{nd} unit operation 407 may be a distillation column, which separates the 1,2,2,3-pentachloropropane (HCC-240aa), hydrogen fluoride (HF), and the azeotrope or azeotrope like composition of 1,2,2,3-pentachloropropane (HCC-240aa) and hydrogen fluoride (HF) into overhead stream 416, and any one of the additional components of the product stream 414, such as the 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), 1,3-dichloro-3,3-difluoro-1-propene (HFC-1232zd) isomers into recovery stream 418.

Although not illustrated, recovery stream 418 may be sent to a further separation unit to separate the 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), and/or cis-1-chloro-2,3,3-trifluoroprop-1-ene (HCFO-1233 yd(Z)), which may be further reacted, such as via reactions steps (ii) and (iii) described previously, to produce 2,3,3,3-tetrafluoro-I-propene (HFO-1234yf).

Overhead stream 416 may optionally be fed into 3rd unit operation 409, which may be used to separate any one of the components of overhead stream 416 from one another. For instance, any one of the 1,2,2,3-pentachloropropane (HCC-240aa), hydrogen fluoride (HF), and the azeotrope or azeotrope like composition of 1,2,2,3-pentachloropropane (HCC-240aa) and hydrogen fluoride (HF) may be separated from each other and/or third unit operation 409 may be used to “break” the azeotrope or azeotrope-like composition of 1,2,2,3-pentachloropropane (HCC-240aa) and hydrogen fluoride (HF) into individual components by known techniques, such as by pressure swing distillation or extractive distillation and the like. Optional Recovery stream 422 may therefore comprise either the 1,2,2,3-pentachloropropane (HCC-240aa) or hydrogen fluoride (HF) and recycle stream 420 may comprise either hydrogen fluoride (HF) or 1,2,2,3-pentachloropropane (HCC-240aa), respectively. Recycle stream 420 is thereafter recycled and fed back into 1^{st} unit operation 405 as a reactant comprising any one of 1,2,2,3-pentachloropropane (HCC-240aa), hydrogen fluoride (HF), and/or the azeotrope or azeotrope-like composition of 1,2,2,3-pentachloropropane (HCC-240aa) and hydrogen fluoride (HF).

In the case where optional 3^{rd} unit operation 409 is not used, overhead stream 416 is the same as recycle stream 420, comprising any one of the 1,2,2,3-pentachloropropane (HCC-240aa) and hydrogen fluoride (HF) and/or the azeotrope or azeotrope-like composition of 1,2,2,3-pentachloropropane (HCC-240aa), and optional recovery stream 422 is not recovered.

### Catalysts

The catalyst plays an important role in the reaction. Suitable catalysts useful in the fluorination reaction include: (I) pentavalent antimony, niobium, arsenic and tantalum halides; (II) pentavalent antimony, niobium, arsenic and tantalum mixed halides; and (III) mixtures of pentavalent antimony, niobium, arsenic and tantalum halide catalysts. Examples of catalysts of group (I) include antimony pentachloride and antimony pentafluoride. Examples of catalysts of group (II) include SbCl₂F₃ and SbBr₂F₃. Examples of catalysts of group (III) include a mixture of antimony pentachloride and antimony pentafluoride.

Suitable catalysts may also include chromium oxides, chromium oxyfluorides, and chromium halides. The chromium oxides may include amorphous chromium oxide (Cr₂O₃), crystalline chromium oxide, and combinations of the foregoing. The chromium oxyfluorides may include fresh amorphous chromium oxide (Cr₂O₃) pretreated with HF, fresh crystalline chromium oxide (Cr₂O₃) pretreated with HF, amorphous chromium oxyfluoride (CrOₓF_{y}, where x may be greater than 0 but less than 1.5, and y may be greater than 0 but less than 3), crystalline chromium oxyfluoride (CrOₓF_{y}, where x may be greater than 0 but less than 1.5, and y may be greater than 0 but less than 3), and combinations of the foregoing. The catalyst may be amorphous chromium oxyfluoride (CrOₓF_{y}, where x may be greater than 0 but less than 1.5, and y may be greater than 0 but less than 3). The chromium halides may include chromium trifluoride (CrF₃), chromium trichloride (CrCl₃), chromium triiodide (CrI₃) and chromium tribromide (CrBr₃), and combinations of the foregoing. For example, the catalyst is chromium trifluoride (CrF₃). Fluorinated chromium oxide may be also used.

Other suitable catalysts include promoted chromium-based catalysts, which are based on chromium and include an amount of at least one co-catalyst selected from Ni, Zn, Co, Mn, Mg, or mixtures thereof. The amount of the co-catalyst may be between 0.1 wt. % and 20 wt. % based on the total weight of the catalyst and, more particularly, may be present in an amount as little as 0.1 wt. %, 0.5 wt. %, 1.0 wt. % 1.5 wt. % or as high as 2.0 wt. %, 3.0 wt. %, 4.0 wt. %, 5.0 wt. %, 6.0 wt. %, or within any range using any two of the foregoing values as endpoints, based on to total weight of the catalyst. One suitable promoted chromium catalyst is a zinc/chromia catalyst which is based on chromia and includes an amount of zinc as a co-catalyst. Prior to use, a fluorination treatment of the catalyst may be conducted using anhydrous HF under conditions effective to convert a portion of metal oxides into corresponding metal fluorides.

The above chromium-based catalysts may also be low chromium (VI) catalysts, having a total content of chromium (VI) oxide in an amount of about 5,000 ppm or less, about 2,000 ppm or less, about 1,000 ppm or less, about 500 ppm or less, about 250 ppm or less, or about 100 ppm or less based on total chromium oxides in the chromium oxide catalyst.

In addition to chromium-based catalysts, other suitable catalysts include alumina, iron oxide, magnesium oxide, zinc oxide, nickel oxide, cobalt oxide, aluminum fluoride or metal fluorides such as iron fluoride, magnesium fluoride, zinc fluoride, nickel fluoride, cobalt fluoride, fluorinated alumina, fluorinated iron oxide, fluorinated magnesium oxide, fluorinated nickel oxide, fluorinated cobalt oxide, titanium fluorides, molybdenum fluorides, aluminum oxyfluorides, and combinations of the foregoing. Prior to use, a fluorination treatment of catalyst containing metal oxide(s) is conducted using anhydrous HF under conditions effective to convert a portion of metal oxide(s) into corresponding metal fluoride(s).

Pentavalent antimony, niobium, arsenic and tantalum halides are commercially available, and mixed halides thereof are created in situ upon reaction with HF. Antimony pentachloride is preferred because of its low cost and availability. Pentavalent antimony mixed halides of the formula SbClₙF₅₋ₙ where n is 0 to 5 are more preferred. The fluorination catalysts preferably have a purity of at least about 97%. Although the amount of fluorination catalyst used may vary widely, using from about 5% to about 50%, or preferably from about 10% to about 25% by weight catalyst, relative to the organics is suitable.

Suitable catalysts for use in the fluorination reaction are listed in Table 1 below. Each of the catalyst/cocatalysts listed in Table 1 may be supported on, for example, activated carbon.

TABLE 1
Catalysts for Fluorination Reaction

| Catalyst | Cocatalyst |
|---|---|
| antimony pentachloride (SbCl₅) | None |
| antimony pentafluoride (SbF₅) | None |
| SbCl₂F₃ | None |
| SbBr₂F₃ | None |
| Mixed SbCl₅ and SbF₅ | None |
| chromium oxide (Cr₂O₃) | None |
| chromium oxyfluorides (CrOₓF_{y}) | None |
| chromium trifluoride (CrF₃) | None |
| chromium trichloride (CrCl₃) | None |
| chromium triiodide (Crl₃) | None |
| chromium tribromide (CrBr₃) | None |
| Cr₂O₃ | Ni |
| Cr₂O₃ | Zn |
| Cr₂O₃ | Co |
| Cr₂O₃ | Mn |
| Cr₂O₃ | Mg |
| fluorinated Cr₂O₃ | None |
| alumina | None |
| iron oxide | None |
| magnesium oxide | None |
| zinc oxide | None |
| nickel oxide | None |
| cobalt oxide | None |
| aluminum fluoride | None |
| iron fluoride | None |
| magnesium fluoride | None |
| zinc fluoride | None |
| nickel fluoride | None |
| cobalt fluoride | None |
| fluorinated alumina | None |
| fluorinated iron oxide | None |
| fluorinated magnesium oxide | None |
| fluorinated nickel oxide | None |
| fluorinated cobalt oxide | None |
| titanium fluoride | None |
| molybdenum fluoride | None |
| aluminum oxyfluoride | None |
| Niobium halide | None |
| Arsenic halide | None |
| Tantalum halide | None |

### Molar Ratio During the Fluorination Reaction, the Reactant Composition Comprising

1,1,2,2,3-pentachloropropane (HCC-240aa) and the hydrogen fluoride (HF) may be fed into the reactor separately or in combination. The molar ratio of hydrogen fluoride to 1,1,2,2,3-pentachloropropane (HCC-240aa) may be as little as about 15, 20, 25, 30, or as great as about 35, 40, 45, 50, or within any range encompassed by any two of the foregoing values as endpoints, for example, from about 15 to about 50, from about 20 to about 50, from about 20 to 40, or from about 25 to 35. The molar ratio of hydrogen fluoride to 1,1,2,2,3-pentachloropropane (HCC-240aa) may be about 32.4. Specific examples of additional suitable ranges are set forth below in Table 2. The numerical ranges set forth in Table 2 below are understood to be prefaced by “about”.

TABLE 2
Molar Ratio of HCC-240aa:HF in Fluorination Reaction

| From | To |
|---|---|
| 15 | 50 |
| 20 | 50 |
| 25 | 50 |
| 30 | 50 |
| 15 | 45 |
| 15 | 40 |
| 15 | 35 |
| 15 | 30 |
| 15 | 20 |
| 20 | 25 |
| 25 | 30 |
| 30 | 35 |
| 35 | 40 |
| 40 | 45 |
| 45 | 50 |

### Reaction Temperature

The reaction temperature for the fluorination reaction may be as low as about 75° C., about 80° C., about 85° C., about 90° C., about 95° C., about 100° C., or as high as about 125° C., about 150° C., about 175° C., about 200° C., about 225° C., about 250° C., about 275° C., about 300° C., about 325° C., about 350° C., or within any range encompassed by any two of the foregoing values as endpoints. For example, the reaction temperature may be from about 75° C. to about 350° C., about 100° C. to about 300° C., or about 150° C. to about 250° C. Specific examples of additional suitable ranges are set forth below in Table 3. The numerical ranges set forth in Table 3 below are understood to be prefaced by “about”.

TABLE 3
Reaction Temperature of Fluorination Reaction

| From (° C.) | To (° C.) |
|---|---|
| 75 | 350 |
| 75 | 325 |
| 75 | 300 |
| 75 | 275 |
| 75 | 250 |
| 75 | 225 |
| 75 | 200 |
| 75 | 175 |
| 75 | 150 |
| 75 | 125 |
| 100 | 350 |
| 100 | 325 |
| 100 | 300 |
| 100 | 250 |
| 100 | 225 |
| 100 | 200 |
| 75 | 100 |
| 100 | 120 |
| 120 | 200 |
| 200 | 225 |
| 225 | 250 |
| 250 | 300 |
| 300 | 325 |
| 325 | 350 |

### Reaction Pressure

The reaction pressure may be as low as about 1 psig, about 2 psig, about 3 psig, about 4 psig, about 5 psig, about 6 psig, about 7 psig, about 8 psig, about 9 psig, about 10 psig, about 11 psig, about 12 psig, about 13 psig, about 14 psig, about 15 psig, about 16 psig, about 17 psig, about 18 psig, about 19 psig, about 20 psig, about 21 psig, about 22 psig, about 23 psig, about 24 psig, about 25 psig, about 26 psig, about 27 psig, about 28 psig, about 29 psig, about 30 psig, or as high as about 100 psig, 110 psig, 120 psig, 130 psig, 140 psig, 150 psig, 160 psig, 170 psig, 180 psig, 190 psig, or 200 psig, or within any range encompassed by any two of the foregoing values as endpoints. For example, the reaction pressure may be from about 1 psig to about 200 psig, from about 5 psig to about 150 psig, or from about 10 psig to about 120 psig. Specific examples of additional suitable ranges are set forth below in Table 4. The numerical ranges set forth in Table 4 below are understood to be prefaced by “about”.

TABLE 4
Reaction Pressure of Fluorination Reaction

| From (psig) | To (psig) |
|---|---|
| 1 | 200 |
| 1 | 180 |
| 1 | 160 |
| 1 | 140 |
| 1 | 120 |
| 10 | 200 |
| 10 | 180 |
| 10 | 160 |
| 10 | 140 |
| 10 | 120 |
| 1 | 5 |
| 5 | 10 |
| 10 | 20 |
| 20 | 50 |
| 50 | 100 |
| 100 | 120 |
| 120 | 150 |
| 150 | 200 |

A summary of the preferred catalyst, molar ratio, reaction pressure, and reaction temperatures as discussed above are summarized in Table 5 below. The numerical ranges set forth in Table 5 below are understood to be prefaced by “about”. Each of the catalysts set forth in Table 5 below may be supported on activated carbon. The fluorinated chromium oxide may be zinc promoted fluorinated chromium oxide.

TABLE 5
Summary of Catalyst and Reaction Conditions

| | Molar Ratio | Pressure | Temperature |
|---|---|---|---|
| Catalyst | (HCC-240aa:HF) | (psig) | (° C.) |
| fluorinated Cr₂O₃ | 15-50 | 10-200 | 75-350 |
| fluorinated Cr₂O₃ | 15-50 | 10-200 | 100-300 |
| fluorinated Cr₂O₃ | 15-50 | 15-150 | 75-350 |
| fluorinated Cr₂O₃ | 15-50 | 15-150 | 100-300 |
| fluorinated Cr₂O₃ | 20-40 | 10-200 | 75-350 |
| fluorinated Cr₂O₃ | 20-40 | 10-200 | 100-300 |
| fluorinated Cr₂O₃ | 20-40 | 15-150 | 75-350 |
| fluorinated Cr₂O₃ | 20-40 | 15-150 | 100-300 |
| fluorinated Al₂O₃ | 15-50 | 10-200 | 75-350 |
| fluorinated Al₂O₃ | 15-50 | 10-200 | 100-300 |
| fluorinated Al₂O₃ | 15-50 | 15-150 | 75-350 |
| fluorinated Al₂O₃ | 15-50 | 15-150 | 100-300 |
| fluorinated Al₂O₃ | 20-40 | 10-200 | 75-350 |
| fluorinated Al₂O₃ | 20-40 | 10-200 | 100-300 |
| fluorinated Al₂O₃ | 20-40 | 15-150 | 75-350 |
| fluorinated Al₂O₃ | 20-40 | 15-150 | 100-300 |
| SbCl₅ | 15-50 | 10-200 | 75-350 |
| SbCl₅ | 15-50 | 10-200 | 100-300 |
| SbCl₅ | 15-50 | 15-150 | 75-350 |
| SbCl₅ | 15-50 | 15-150 | 100-300 |
| SbCl₅ | 20-40 | 10-200 | 75-350 |
| SbCl₅ | 20-40 | 10-200 | 100-300 |
| SbCl₅ | 20-40 | 15-150 | 75-350 |
| SbCl₅ | 20-40 | 15-150 | 100-300 |
| SbClₙF₅₋ₙ (n = 0-5) | 15-50 | 10-200 | 75-350 |
| SbClₙF₅₋ₙ (n = 0-5) | 15-50 | 10-200 | 100-300 |
| SbClₙF₅₋ₙ (n = 0-5) | 15-50 | 15-150 | 75-350 |
| SbClₙF₅₋ₙ (n = 0-5) | 15-50 | 15-150 | 100-300 |
| SbClₙF₅₋ₙ (n = 0-5) | 20-40 | 10-200 | 75-350 |
| SbClₙF₅₋ₙ (n = 0-5) | 20-40 | 10-200 | 100-300 |
| SbClₙF₅₋ₙ (n = 0-5) | 20-40 | 15-150 | 75-350 |
| SbClₙF₅₋ₙ (n = 0-5) | 20-40 | 15-150 | 100-300 |

### Product Mixture

The product mixture may comprise 2-chloro-3,3,3-trifluoroprop-1-ene (HCFO-1233xf) in an amount of as little as about 1 wt. %, about 14.6 wt. %, or about 27.2 wt. % or as great as 39.8 wt. %, 52.4 wt. % or 65 wt. %, or within any range including any two of the foregoing amounts as endpoints, based on the total weight of the product mixture.

The product mixture may comprise HCFO-1233zd(E) in an amount of as little as about 0.0 wt. %, about 0.6 wt. %, or about 1.2 wt. % or as great as 1.8 wt. %, 2.4 wt. % or 3.0 wt. %, or within any range including any two of the foregoing amounts as endpoints, based on the total weight of the product mixture.

The product mixture may comprise HCFO-1233zd(Z) in an amount of as little as about 0.0 wt. %, about 1.4 wt. %, or about 2.8 wt. % or as great as 4.2 wt. %, 5.6 wt. % or 7.0 wt. %, or within any range including any two of the foregoing amounts as endpoints, based on the total weight of the product mixture.

The product mixture may comprise Cis-1-Chloro-2,3,3-trifluoroprop-1-ene (HCFO-1233 yd(Z)) in an amount of as little as about 0.0 wt. %, about 2.4 wt. %, or about 4.8 wt. % or as great as 7.2 wt. %, 9.6 wt. % or 12.0 wt. %, or within any range including any two of the foregoing amounts as endpoints, based on the total weight of the product mixture.

The product mixture may comprise 1,3-dichloro-3,3-difluoro-1-propene (HFC-1232zd) isomers in a total amount of as little as about 4.0 wt. %, about 12.2 wt. %, or about 20.4 wt. % or as great as 28.6 wt. %, 36.8 wt. % or 45.0 wt. %, or within any range including any two of the foregoing amounts as endpoints, based on the total weight of the product mixture.

The product mixture may comprise 1,1,2,2,3-pentachloropropane (HCC-240aa) in an amount of as little as about 0.0 wt. %, about 7.0 wt. %, or about 14.0 wt. % or as great as 21.0 wt. %, 28.0 wt. % or 35.0 wt. %, or within any range including any two of the foregoing amounts as endpoints, based on the total weight of the product mixture.

The foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### EXAMPLES

### PTx—Binary Method:

For the Example below, saturation pressure was measured using a PTx cell consisting of a nominal volume of 40 mL, equipped with a calibrated pressure transducer, a resistance temperature detector (RTD), and valving to permit the transfer of components. A thermostatted temperature bath was used to maintain the PTx cell's isotherm, capable of fixing the temperature between 0 to 80° C.

The PTx measurement was carried out by first introducing about 8.2 g of 1,1,2,2,3-pentachloropropane (HCC-240aa) having a purity of >99 area % as determined by gas chromatography (GC). The cell was brought to saturation by adjusting the temperature bath to about 20° C., 40° C. or 60° C., establishing an equilibrium pressure of the 1,1,2,2,3-pentachloropropane (HCC-240aa) which was recorded. Then, anhydrous hydrogen fluoride (HF) was introduced into the PTx cell in small, measured, gravimetric increments. After a predetermined amount of hydrogen fluoride (HF) was added to the PTx cell, the system was allowed to reach equilibrium for approximately 1 to 3 hours before the equilibrium pressure of the mixture was recorded. Gravimetric additions of hydrogen fluoride (HF) were successively added to the cell to adjust the composition in the PTx cell, permitting a new saturation pressure to be observed. Once the PTx cell was effectively full because of these additions, the system was reset, restarting with an initial charge of about 14.6 g of hydrogen fluoride (HF), and subsequent introductions of 1,1,2,2,3-pentachloropropane (HCC-240aa).

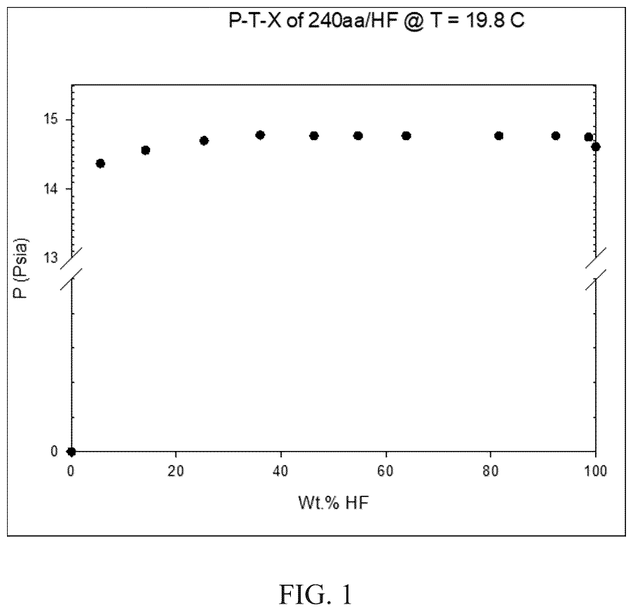

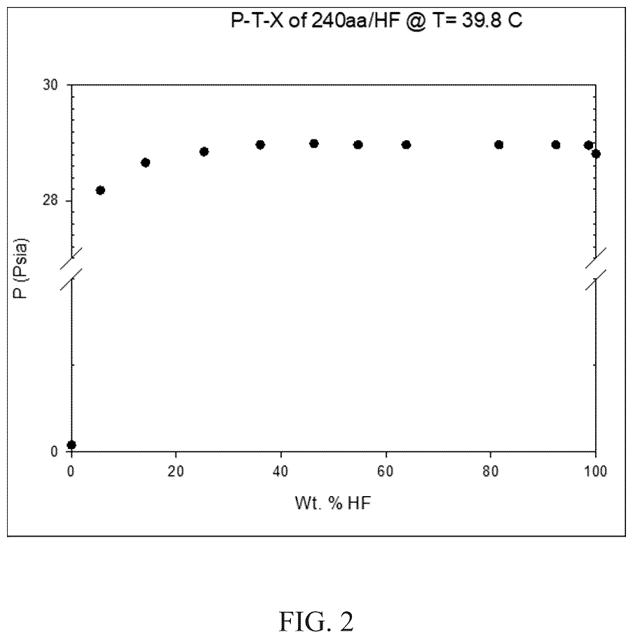

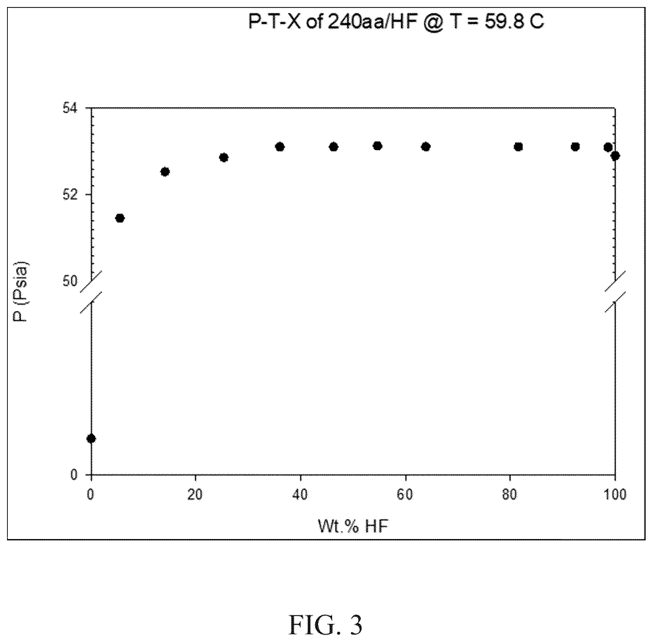

Through the PTx methodology, composition versus saturated pressure data as a function of temperature were obtained for the composition range from 0 to 100 weight percent of hydrogen fluoride (HF) and is presented in the table below which shows a maximum in pressure, indicating that a minimum boiling, heterogeneous azeotrope had been formed. These data are also shown FIGS. 1, 2, and 3.

### Example 1: PTx Study with 1,1,2,2,3-Pentachloropropane (HCC-240aa) and Hydrogen Fluoride (HF)

Table 6 shows the saturation pressure measurements of a binary HCC-240aa/HF mixture as a function of composition with varying weight percent hydrogen fluoride (HF) at constant temperatures of about 20° C., 40° C. and 60° C., measured via PTx.

TABLE 6
Compositions of 1,1,2,2,3-Pentachloropropane (HCC-240aa)
and Hydrogen Fluoride (HF) vs. Vapor Pressure

| | HF | HCC-240aa | Saturation |
|---|---|---|---|
| Temperature | Composition | Composition | Pressure |
| (° C.) | (wt. %) | (wt. %) | (psia) |
| 19.8 ± 0.2 | 100.00 | 0.00 | 14.61 |
| | 98.63 | 1.37 | 14.75 |
| | 92.36 | 7.64 | 14.77 |
| | 81.51 | 18.49 | 14.77 |
| | 63.84 | 36.16 | 14.77 |
| | 54.66 | 45.34 | 14.77 |
| | 46.26 | 53.74 | 14.77 |
| | 35.99 | 64.01 | 14.78 |
| | 25.30 | 74.7 | 14.70 |
| | 14.12 | 85.88 | 14.56 |
| | 5.51 | 94.49 | 14.37 |
| | 0.00 | 100.00 | 0.00 |
| 39.8 ± 0.2 | 100.00 | 0.00 | 28.81 |
| | 98.63 | 1.37 | 28.96 |
| | 92.36 | 7.64 | 28.97 |
| | 81.51 | 18.49 | 28.97 |
| | 63.84 | 36.16 | 28.97 |
| | 54.66 | 45.34 | 28.97 |
| | 46.26 | 53.74 | 28.99 |
| | 35.99 | 64.01 | 28.97 |
| | 25.30 | 74.7 | 28.85 |
| | 14.12 | 85.88 | 28.66 |
| | 5.51 | 94.49 | 28.18 |
| | 0.00 | 100.00 | 0.04 |
| 59.8 ± 0.2 | 100.00 | 0.00 | 52.90 |
| | 98.63 | 1.37 | 53.10 |
| | 92.36 | 7.64 | 53.11 |
| | 81.51 | 18.49 | 53.11 |
| | 63.84 | 36.16 | 53.11 |
| | 54.66 | 45.34 | 53.13 |
| | 46.26 | 53.74 | 53.11 |
| | 35.99 | 64.01 | 53.11 |
| | 25.30 | 74.7 | 52.86 |
| | 14.12 | 85.88 | 52.53 |
| | 5.51 | 94.49 | 51.46 |
| | 0.00 | 100.00 | 0.21 |

The data shows that the mixture is a minimum boiling heterogeneous azeotropic or azeotrope-like mixture since the saturation pressure of the binary mixtures of HCC-240aa/HF are higher at all indicated blend proportions compared to the saturation pressure of either pure component.

In view of the above data, identification the of Vapor-Liquid-Liquid equilibrium (VLLE) miscibility gap was applied to determine the azeotrope and azeotrope-like compositions.

The VLLE miscibility gap is defined by a range of overall compositions for which individual liquid phases and vapor phase compositions remains constant. The miscibility gap may be derived from thermodynamic measurements, such as those collected via PTx methods, subject to material balance and thermodynamic constraints. Several methods for deriving the miscibility gap from thermodynamic measurements are described in Sandler, S. I. (2006). Chapter 11: Other Types of Phase Equilibria in Fluid Mixtures. In Chemical, Biochemical, and Engineering Thermodynamics (4^{th} ed., pp. 575-657) which includes constraining thermodynamic consistency through the fundamental Gibbs-Duhem relationship and resolving the vapor phase composition, from the measurements, through combined mass balance and equilibrium criteria (frequently referred to as the Rachford-Rice equation or algorithm). Through this derivation, the relationship between equilibrium compositions, temperatures, and pressures are established permitting the VLLE miscibility gap to be evaluated.

For a given temperature, the miscibility gap can be identified as compositions that yield a constant saturation pressure at thermodynamic equilibrium. Within the miscibility gap, the compositions of the three individual phases will remain constant which includes the azeotropic composition; a composition where the aggregated liquid composition is equal to the vapor composition. Given that saturation pressure remains constant, upon heating, compositions within the miscibility gap will remain also constant, only yielding changes to the total amount (e.g. volume) each phase. As such, it has been identified that compositions within the miscibility gap are considered azeotrope-like.

Based on the above, the azeotrope-like composition may comprise from about 0.1 wt. % to 78.9 wt. % 1,1,2,2,3-pentachloropropane (HCC-240aa), preferably from about 0.2 wt. % to 72.5 wt. % 1,1,2,2,3-pentachloropropane (HCC-240aa), or preferably from about 0.6 wt. % to 64.0 wt. % 1,1,2,2,3-pentachloropropane (HCC-240aa) and from about 21.1 wt. % to 99.9 wt. % hydrogen fluoride (HF), preferably from about 27.5 wt. % to 99.8 wt. % hydrogen fluoride (HF), or preferably from about 36.0 wt. % to 99.4 wt. % hydrogen fluoride (HF).

The true azeotrope is about 9.5 wt. % 1,1,2,2,3-pentachloropropane (HCC-240aa) and 90.5 wt. % hydrogen fluoride (HF) and has a boiling point of about 19° C. ±0.01° C. at a pressure of about 14.7 psia±0.2 psia.

In other words, the azeotrope or azeotrope-like composition consists essentially of from about 0.1 wt. % to 78.9 wt. % of 1,1,2,2,3-pentachloropropane (HCC-240aa) and 21.1 wt. % to 99.9 wt. % hydrogen fluoride (HF), from about 0.2 wt. % to 72.5 wt. % 1,1,2,2,3-pentachloropropane (HCC-240aa) and 27.5 wt. % to 99.8 wt. % hydrogen fluoride (HF), from about 0.6 wt. % to 64.0 wt. % 1,1,2,2,3-pentachloropropane (HCC-240aa) and 36.0 wt. % to 99.4 wt. % hydrogen fluoride (HF), or about 9.5 wt. % 1,1,2,2,3-pentachloropropane (HCC-240aa) and 90.5 wt. % hydrogen fluoride (HF). In some cases, the azeotrope or azeotrope-like composition consists of from about 0.1 wt. % to 78.9 wt. % of 1,1,2,2,3-pentachloropropane (HCC-240aa) and 21.1 wt. % to 99.9 wt. % hydrogen fluoride (HF), from about 0.2 wt. % to 72.5 wt. % 1,1,2,2,3-pentachloropropane (HCC-240aa) and 27.5 wt. % to 99.8 wt. % hydrogen fluoride (HF), from about 0.6 wt. % to 64.0 wt. % 1,1,2,2,3-pentachloropropane (HCC-240aa) and 36.0 wt. % to 99.4 wt. % hydrogen fluoride (HF), or about 9.5 wt. % 1,1,2,2,3-pentachloropropane (HCC-240aa) and 90.5 wt. % hydrogen fluoride (HF)

### Example 2: Vapor-Liquid-Liquid Equilibrium (VLLE) Measurement with 1,1,2,2,3-Pentachloropropane (HCC-240aa) and Hydrogen Fluoride (HF)

The azeotropic or azeotrope-like composition of the binary HCC-240aa/HF was also verified by Vapor-Liquid-Liquid Equilibrium (VLLE) measurement. A mixture of 60.06 grams of 1,1,2,2,3-pentachloropropane (HCC-240aa) and 29.94 grams of hydrogen fluoride (HF) was blended to form a heterogeneous solution (2 phases by visual observation) in a Teflon cell. The composition of this mixture contained 66.73 1,1,2,2,3-pentachloropropane (HCC-240aa) and 33.27% hydrogen fluoride (HF). The vapor of this mixture was sampled at 19° C. and determined to be 90.5±2 wt. %.

### Example 3: Process for Co-producing Trifluorochloropropenes from 1,1,2,2,3-Pentachloropropane (HCC-240aa) and Hydrogen Fluoride (HF)

In the following examples, the process of producing trifluorochloropropenes (HCFO-1233) from 240aa and anhydrous HF is demonstrated. A ¾″ Inconel 600 tubular reactor (0.0625″ wall thickness and 36″ long) was charged with 100 ml of solid catalyst selected from fluorinated chromium oxide, zinc promoted fluorinated chromium oxide, fluorinated Al₂O₃ and 50% SbCl₅ supported by activated carbon. After the reactor was preheated to a designated reaction temperature, certain amount of 240aa and HF was co-fed into a vaporizer with the flowrates controlled by mass flowmeters, and then the vapor was fed into the heated fixed bed tubular reactor to have the reaction conducted in the catalyst bed. A control valve was used at the reactor outlet to control the reaction pressure. Periodically, samples were taken from the effluent of the reactor, and the composition of the organic compounds in the samples were measured by GC and/or GCMS.

With fluorinated chromium oxide as the catalyst, at 200° C. of the reactor temperature and 16 psig of reactor pressure, 7.6 g/h of HCC-240aa and 25.7 g/h of HF, which gave a HF/240aa molar ratio of 32.4, were co-fed into the reactor. By GCMS, the reactor effluent composition is shown in the table below:

TABLE 7
GC area % for trifluorochloropropenes

| Component | GC (area %) |
|---|---|
| 2-chloro-3,3,3-trifluoroprop-1-ene (HCFO-1233xf) | 39.28 |
| trans-1-chloro-3,3,3-trifluoropropene (HCFO-1233zd(E)) | 0.02 |
| cis-1-chloro-3,3,3-trifluoropropene (HCFO-1233zd(Z)) | 1.39 |
| cis-1-Chloro-2,3,3-trifluoroprop-1-ene (HCFO-1233yd(Z)) | 5.53 |
| 1,3-dichloro-3,3-difluoro-1-propene (HFC-1232zd) isomers | 43.96 |
| 1,1,2,2,3-pentachloropropane (HCC-240aa) | 3.15 |
| Others | 6.67 |

### Example 4: Process for Co-producing Trifluorochloropropenes from 1,1,2,2,3-Pentachloropropane (HCC-240aa) and Hydrogen Fluoride (HF)

With fluorinated chromium oxide as the catalyst, at 250° C. of the reactor temperature and 16 psig of reactor pressure, 9.1 g/h of 240aa and 25.7 g/h of HF, which gave a HF/240aa molar ratio of 30.6, were co-fed into the reactor. By GCMS, the reactor effluent composition is shown in the table below:

TABLE 8
GC area % for trifluorochloropropenes

| Component | GC (area %) |
|---|---|
| 2-chloro-3,3,3-trifluoroprop-1-ene (HCFO-1233xf) | 52.33 |
| trans-1-chloro-3,3,3-trifluoropropene (HCFO-1233zd(E)) | 0.02 |
| cis-1-chloro-3,3,3-trifluoropropene (HCFO-1233zd(Z)) | 6.83 |
| cis-1-Chloro-2,3,3-trifluoroprop-1-ene (HCFO-1233yd(Z)) | 2.33 |
| 1,3-dichloro-3,3-difluoro-1-propene (HFC-1232zd) isomers | 28.95 |
| 1,1,2,2,3-pentachloropropane (HCC-240aa) | 0.53 |
| Others | 9.01 |

### Example 5: Process for Co-producing Trifluorochloropropenes from 1,1,2,2,3-Pentachloropropane (HCC-240aa) and Hydrogen Fluoride (HF)

With zinc promoted fluorinated chromium oxide as the catalyst, at 200° C. of the reactor temperature and 16 psig of reactor pressure, 9.6 g/h of 240aa and 24.1 g/h of HF, which gave a HF/240aa molar ratio of 27.0, were co-fed into the reactor. By GCMS, the reactor effluent composition is shown in the table below:

TABLE 9
GC area % for trifluorochloropropenes

| Component | GC (area %) |
|---|---|
| 2-chloro-3,3,3-trifluoroprop-1-ene (HCFO-1233xf) | 29.70 |
| trans-1-chloro-3,3,3-trifluoropropene (HCFO-1233zd(E)) | 2.18 |
| cis-1-chloro-3,3,3-trifluoropropene (HCFO-1233zd(Z)) | 0.40 |
| cis-1-Chloro-2,3,3-trifluoroprop-1-ene (HCFO-1233yd(Z)) | 10.35 |
| 1,3-dichloro-3,3-difluoro-1-propene (HFC-1232zd) isomers | 27.24 |
| 1,1,2,2,3-pentachloropropane (HCC-240aa) | 1.54 |
| Others | 28.59 |

### Example 6: Process for Co-producing Trifluorochloropropenes from 1,1,2,2,3-Pentachloropropane (HCC-240aa) and Hydrogen Fluoride (HF)

With zinc promoted fluorinated chromium oxide as the catalyst, at 250° C. of the reactor temperature and 16 psig of reactor pressure, 9.5 g/h of 240aa and 25 g/h of HF, which gave a HF/240aa molar ratio of 28.5, were co-fed into the reactor. By GCMS, the reactor effluent composition is shown in the table below:

TABLE 10
GC area % for trifluorochloropropenes

| Component | GC (area %) |
|---|---|
| 2-chloro-3,3,3-trifluoroprop-1-ene (HCFO-1233xf) | 62.39 |
| trans-1-chloro-3,3,3-trifluoropropene (HCFO-1233zd(E)) | 1.67 |
| cis-1-chloro-3,3,3-trifluoropropene (HCFO-1233zd(Z)) | 4.94 |
| cis-1-Chloro-2,3,3-trifluoroprop-1-ene (HCFO-1233yd(Z)) | 0.00 |
| 1,3-dichloro-3,3-difluoro-1-propene (HFC-1232zd) isomers | 21.32 |
| 1,1,2,2,3-pentachloropropane (HCC-240aa) | 0.00 |
| Others | 9.68 |

### Example 7: Process for Co-producing Trifluorochloropropenes from 1,1,2,2,3-Pentachloropropane (HCC-240aa) and Hydrogen Fluoride (HF)

With Fluorinated Al₂O₃ as the catalyst, at 200° C. of the reactor temperature and 16 psig of reactor pressure, 9 g/h of 240aa and 25.8 g/h of HF, which gave a HF/240aa molar ratio of 31.1, were co-fed into the reactor. By GCMS, the reactor effluent composition is shown in the table below:

TABLE 11
GC area % for trifluorochloropropenes

| Component | GC (area %) |
|---|---|
| 2-chloro-3,3,3-trifluoroprop-1-ene (HCFO-1233xf) | 2.53 |
| Trans-1-chloro-3,3,3-trifluoropropene (HCFO-1233zd(E)) | 0.55 |
| Cis-1-chloro-3,3,3-trifluoropropene (HCFO-1233zd(Z)) | 0.60 |
| Cis-1-Chloro-2,3,3-trifluoroprop-1-ene (HCFO-1233yd(Z)) | 0 |
| 1,3-dichloro-3,3-difluoro-1-propene (HFC-1232zd) isomers | 5.36 |
| 1,1,2,2,3-pentachloropropane (HCC-240aa) | 30.46 |
| Others | 60.50 |

### Example 8: Process for Co-producing Trifluorochloropropenes from 1,1,2,2,3-Pentachloropropane (HCC-240aa) and Hydrogen Fluoride (HF)

With Fluorinated Al₂O₃ as the catalyst, at 250° C. of the reactor temperature and 16 psig of reactor pressure, 6.3 g/h of 240aa and 18 g/h of HF, which gave a HF/240aa molar ratio of 31.1, were co-fed into the reactor. By GCMS, the reactor effluent composition is shown in the table below:

TABLE 12
GC area % for trifluorochloropropenes

| Component | GC (area %) |
|---|---|
| 2-chloro-3,3,3-trifluoroprop-1-ene (HCFO-1233xf) | 2.45 |
| trans-1-chloro-3,3,3-trifluoropropene (HCFO-1233zd(E)) | 0.14 |
| cis-1-chloro-3,3,3-trifluoropropene (HCFO-1233zd(Z)) | 0 |
| cis-1-Chloro-2,3,3-trifluoroprop-1-ene (HCFO-1233yd(Z)) | 0 |
| 1,3-dichloro-3,3-difluoro-1-propene (HFC-1232zd) isomers | 19.36 |
| 1,1,2,2,3-pentachloropropane (HCC-240aa) | 24.70 |
| Others | 53.34 |

### Example 9: Process for Co-producing Trifluorochloropropenes from 1,1,2,2,3-Pentachloropropane (HCC-240aa) and Hydrogen Fluoride (HF)

With Fluorinated Al₂O₃ as the catalyst, at 300° C. of the reactor temperature and 16 psig of reactor pressure, 9.3 g/h of 240aa and 26.4 g/h of HF, which gave a HF/240aa molar ratio of 30.8, were co-fed into the reactor. By GCMS, the reactor effluent composition is shown in the table below:

TABLE 13
GC area % for trifluorochloropropenes

| Component | GC (area %) |
|---|---|
| 2-chloro-3,3,3-trifluoroprop-1-ene (HCFO-1233xf) | 10.18 |
| trans-1-chloro-3,3,3-trifluoropropene (HCFO-1233zd(E)) | 0.12 |
| cis-1-chloro-3,3,3-trifluoropropene (HCFO-1233zd(Z)) | 1.76 |
| cis-1-Chloro-2,3,3-trifluoroprop-1-ene (HCFO-1233yd(Z)) | 5.84 |
| 1,3-dichloro-3,3-difluoro-1-propene (HFC-1232zd) isomers | 42.43 |
| 1,1,2,2,3-pentachloropropane (HCC-240aa) | 5.21 |
| Others | 34.46 |

### Example 10: Process for Co-producing Trifluorochloropropenes from 1,1,2,2,3-Pentachloropropane (HCC-240aa) and Hydrogen Fluoride (HF)

With 50% SbCl₅ supported by activated carbon as the catalyst, at 95° C. of the reactor temperature and 16 psig of reactor pressure, 9.1 g/h of 240aa and 24 g/h of HF, which gave a HF/240aa molar ratio of 28.6, were co-fed into the reactor. By GCMS, the reactor effluent composition is shown in the table below:

TABLE 14
GC area % for trifluorochloropropenes

| Component | GC (area %) |
|---|---|
| 2-chloro-3,3,3-trifluoroprop-1-ene (HCFO-1233xf) | 18.28 |
| trans-1-chloro-3,3,3-trifluoropropene (HCFO-1233zd(E)) | 0.09 |
| cis-1-chloro-3,3,3-trifluoropropene (HCFO-1233zd(Z)) | 0 |
| cis-1-Chloro-2,3,3-trifluoroprop-1-ene (HCFO-1233yd(Z)) | 0 |
| 1,3-dichloro-3,3-difluoro-1-propene (HFC-1232zd) isomers | 38.86 |
| 1,1,2,2,3-pentachloropropane (HCC-240aa) | 1.74 |
| Others | 30.76 |

### Example 11: Process for producing 2,3,3,3-tetrafluoro-1-propene (HFO-1234yf) from 1,1,2,2,3-pentachloropropane (HCC-240aa) and HF

A reactant stream comprising 1,1,2,2,3-pentachloropropane (HCC-240aa) is charged into a reactor along with Hydrogen Fluoride (HF). The HF and the 1,1,2,2,3-pentachloropropane (HCC-240aa) are reacted, in the presence of a catalyst, to produce a product mixture that contains any one of (a) trifluorochloropropenes including 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), and/or (b) difluorodichloropropenes including 1,3-dichloro-3,3-difluoro-1-propene (HFC-1232zd). The product mixture is then provided to a liquid phase reactor, in the presence of a liquid hydrofluorination catalyst, along with Hydrogen Fluoride (HF). The product mixture and the HF react to form 2-chloro-1,1,1,2-tetrafluoropropane (HCFC-244bb). The 2-chloro-1,1,1,2-tetrafluoropropane (HCFC-244bb) is further provided to a vapor phase reactor. The 2-chloro-1,1,1,2-tetrafluoropropane (HCFC-244bb) is reacted and produces a final reaction product including 2,3,3,3-tetrafluoro-1-propene (HFO-1234yf).

### ASPECTS

Aspect 1 is a composition comprising an azeotrope or azeotrope-like composition consisting essentially of effective amounts of 1,1,2,2,3-pentachloropropane (HCC-240aa) and hydrogen fluoride (HF), wherein the azeotrope or azeotrope-like composition has a boiling point of about 19° C. at a pressure of about 14.7 psia±0.2 psia.

Aspect 2 is a composition according to any proceeding aspect, wherein the azeotrope or azeotrope-like composition consists essentially of from about 0.1 wt. % to about 78.9 wt. % 1,1,2,2,3-pentachloropropane (HCC-240aa) and from about 21.1 wt. % to about 99.9 wt. % hydrogen fluoride (HF).

Aspect 3 is a composition according to any proceeding aspect, wherein the azeotrope or azeotrope-like composition consists essentially of from about 0.2 wt. % to 72.5 wt. % 1,1,2,2,3-pentachloropropane (HCC-240aa) and from about 27.5 wt. % to about 99.8 wt. % hydrogen fluoride (HF).

Aspect 4 is a composition according to any proceeding aspect, wherein the azeotrope or azeotrope-like composition consists essentially of from about 9.5 wt. % 1,1,2,2,3-pentachloropropane (HCC-240aa) and about 90.5 wt. % hydrogen fluoride (HF).

Aspect 5 is a process to produce trifluorochloropropenes comprising the steps of: providing a reactant composition comprising 1,1,2,2,3-pentachloropropane (HCC-240aa); and fluorinating the 1,1,2,2,3-pentachloropropane (HCC-240aa) with hydrogen fluoride (HF) and a catalyst to produce a product mixture.

Aspect 6 is a process according to aspects 5, wherein the molar ratio of hydrogen fluoride (HF) to 1,1,2,2,3-pentachloropropane (HCC-240aa) is from 15 to 50.

Aspect 7 is a process according to any one of aspects 5 through 6, wherein the molar ratio of hydrogen fluoride (HF) to 1,1,2,2,3-pentachloropropane (HCC-240aa) is from 20 to 40.

Aspect 8 is a process according to any one of aspects 5 through 7 wherein the product mixture comprises: about 38 wt. % 2-chloro-3,3,3-trifluoroprop-1-ene (HCFO-1233xf); about 1 wt. % HCFO-1233zd(E); about 1 wt. % HCFO-1233zd(Z); about 5 wt. % Cis-1-chloro-2,3,3-trifluoroprop-1-ene (HCFO-1233 yd(Z)); about 44 wt. % 1,3-dichloro-3,3-difluoro-1-propene (HFC-1232zd) isomers; and about 3 wt. % 1,1,2,2,3-pentachloropropane (HCC-240aa).

Aspect 9 is a process according to any one of aspects 5 through 8, wherein the catalyst is selected from the group consisting of fluorinated chromium oxide, fluorinated alumina, pentavalent antimony halide, niobium halide, arsenic halide, tantalum halide, and pentavalent antimony mixed halides supported on activated carbon.

Aspect 10 is a process according to any one of aspects 5 through 9, wherein the fluorinating step is performed at a temperature of about 75° C. to about 350° C.

Aspect 11 is a process according to any one of aspects 5 through 10, wherein the fluorinating step is performed at a pressure of about 10 psig to about 200 psig.

Aspect 12 is a process according to any one of aspects 5 through 11, wherein one or both of the reactant composition and the hydrogen fluoride (HF) are in the gas phase or liquid phase.

Aspect 13 is a process for the production of trifluorochloropropenes comprising the steps of: providing a reactant composition comprising 1,1,2,2,3-pentachloropropane (HCC-240aa); fluorinating the 1,1,2,2,3-pentachloropropane (HCC-240aa) with hydrogen fluoride (HF) and a catalyst to produce a product mixture comprising an azeotrope or azeotrope-like composition of 1,1,2,2,3-pentachloropropane (HCC-240aa) and hydrogen fluoride (HF); separating the azeotrope or azeotrope-like composition of 1,1,2,2,3-pentachloropropane (HCC-240aa) and hydrogen fluoride (HF) from the product mixture; and recycling the azeotrope or azeotrope-like composition back into the reactant composition.

Aspect 14 is a process for producing 2,3,3,3-tetrafluoro-1-propene (HFO-1234yf) comprising the steps of: providing a reactant composition comprising 1,1,2,2,3-pentachloropropane (HCC-240aa); fluorinating the 1,1,2,2,3-pentachloropropane (HCC-240aa) with hydrogen fluoride (HF) and a catalyst to produce a product mixture, the product mixture comprising: 2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), and/or 1,3-dichloro-3,3-difluoro-1-propene (HFC-1232zd) isomers; fluorinating the product mixture in the presence of a catalyst to produce 2-chloro-1,1,1,2-tetrafluoropropane (HCFC-244bb); and reacting the 2-chloro-1, 1,1,2-tetrafluoropropane (HCFC-244bb) to produce 2,3,3,3-tetrafluoro-1-propene (HFO-1234yf).

Aspect 15 is a composition comprising 2,3,3,3-tetrafluoro-1-propene (HFO-1234yf) produced by the process of Aspect 14.

Aspect 16 is the process of Aspects 11 or 12, wherein the catalyst is selected from the group consisting of fluorinated chromium oxide, fluorinated alumina, and pentavalent antimony mixed halides supported on activated carbon.

The foregoing description is only illustrative of the present disclosure. Various alternatives and modifications can be devised by those skilled in the art without departing from the disclosure. Accordingly, the present disclosure is intended to embrace all such alternatives, modifications, and variances that fall within the scope of the appended claims.

1. A composition comprising an azeotrope or azeotrope-like composition consisting essentially of effective amounts of 1,1,2,2,3-pentachloropropane (HCC-240aa) and hydrogen fluoride (HF), wherein the azeotrope or azeotrope-like composition has a boiling point of about 19° C. at a pressure of about 14.7 psia±0.2 psia.

2. The composition of claim 1, wherein the azeotrope or azeotrope-like composition consists essentially of from about 0.1 wt. % to about 78.9 wt. % 1,1,2,2,3-pentachloropropane (HCC-240aa) and from about 21.1 wt. % to about 99.9 wt. % hydrogen fluoride (HF).

3. The composition of claim 1, wherein the azeotrope or azeotrope-like composition consists essentially of from about 0.2 wt. % to 72.5 wt. % 1,1,2,2,3-pentachloropropane (HCC-240aa) and from about 27.5 wt. % to about 99.8 wt. % hydrogen fluoride (HF).

4. The composition of claim 1, wherein the azeotrope or azeotrope-like composition consists essentially of from about 9.5 wt. % 1,1,2,2,3-pentachloropropane (HCC-240aa) and about 90.5 wt. % hydrogen fluoride (HF).

5. A process to produce trifluorochloropropenes, comprising the steps of:
providing a reactant composition comprising 1,1,2,2,3-pentachloropropane (HCC-240aa); and
fluorinating the 1,1,2,2,3-pentachloropropane (HCC-240aa) with hydrogen fluoride (HF) and a catalyst to produce a product mixture.

6. The process of claim 5, wherein a molar ratio of hydrogen fluoride (HF) to 1,1,2,2,3-pentachloropropane (HCC-240aa) is from 15 to 50.

7. The process of claim 5, wherein the catalyst is selected from the group consisting of fluorinated chromium oxide, fluorinated alumina, pentavalent antimony halide, niobium halide, arsenic halide, tantalum halide, and pentavalent antimony mixed halides supported on activated carbon.

8. The process of claim 5, wherein the fluorinating step is performed at a temperature of about 75° C. to about 350° C.

9. The process of claim 5, wherein the fluorinating step is performed at a pressure of about 10 psig to about 200 psig.

10. The process of claim 5, wherein the reactant composition and the hydrogen fluoride (HF) are in a gas or liquid phase or liquid phase.

11. A process for producing 2,3,3,3-tetrafluoro-1-propene (HFO-1234yf) comprising the steps of:
providing a reactant composition comprising 1,1,2,2,3-pentachloropropane (HCC-240aa);
fluorinating the 1,1,2,2,3-pentachloropropane (HCC-240aa) with hydrogen fluoride (HF) and a catalyst to produce a product mixture, the product mixture comprising:
2-chloro-3,3,3-trifluoroprop-1-ene (HCFO-1233xf), trans-1-chloro-3,3,3-trifluoropropene (HCFO-1233zd(E)), cis-1-chloro-3,3,3-trifluoropropene (HCFO-1233zd(Z)), cis-1-chloro-2,3,3-trifluoroprop-1-ene (HCFO-1233 yd(Z)), and/or 1,3-dichloro-3,3-difluoro-1-propene (HFC-1232zd);
fluorinating the product mixture in the presence of a catalyst to produce 2-chloro-1, 1,1,2-tetrafluoropropane (HCFC-244bb); and
reacting the 2-chloro-1, 1,1,2-tetrafluoropropane (HCFC-244bb) to produce 2,3,3,3-tetrafluoro-I-propene (HFO-1234yf).

12. A composition comprising 2,3,3,3-tetrafluoro-1-propene (HFO-1234yf), produced by the process of claim 11.

13. The process of claim 11, wherein the catalyst is selected from the group consisting of fluorinated chromium oxide, fluorinated alumina, and pentavalent antimony mixed halides supported on activated carbon.
